SEQUENCE LISTING (2 sequences)

SEQ ID NO: 1 - DNA, 495 bp, Homo sapiens
gctcctccaaggctgatctgcgatagcagggtgctggagaggtacctgctggaggctaaggaggctgagaacatcaccaccggctgcgctgagcactgcagcctgaacgagaacatcaccgtgcctgataccaaggtgaacttttacgcttggaagaggatggaggtgggccagcaggctgtggaggtgtggcagggcctggctctgctgagcgaggctgtgctgaggggccaggctctgctggtgaacagctctcagccttgggagcctctgcagctgcacgtggataaggctgtgagcggcctgagaagcctgaccaccctgctgagggctctgggcgctcagaaggaggctatcagccctccagatgctgcaagcgctgcccctctgaggaccatcaccgctgatacctttaggaagctgtttagggtgtacagcaactttctgaggggcaagctgaagctgtacaccggcgaggcttgcaggaccggcgat

SEQ ID NO: 2 - protein, 165 aa, Homo sapiens
APPRLICDSRVLERYLLEAKEAENITTGCAEHCSLNENITVPDTKVNFYAWKRMEVGQQAVEVWQGLALLSEAVLRGQALLVNSSQPWEPLQLHVDKAVSGLRSLTTLLRALGAQKEAISPPDAASAAPLRTITADTFRKLFRVYSNFLRGKLKLYTGEACRTGD

(19) United States
(12) Patent Application Publication
(10) Pub. No.: US 2008/0171696 A1
(43) Pub. Date: Jul. 17, 2008

(54) Pharmacodynamically enhanced therapeutic proteins

(75) Inventor: Kyle D. Yesland, Athens, GA (US)

Correspondence Address: AVIGENICS, INC., 111 RIVERBEND ROAD, ATHENS, GA, 30605, US

(73) Assignee: AviGenics, Inc.

(21) Appl. No.: 12/077,036

(22) Filed: Mar. 14, 2008

Related U.S. Application Data
(63) Continuation-in-part of application No. 11/584,832, filed on Oct. 23, 2006.
(60) Provisional application No. 60/729,429, filed on Oct. 21, 2005.
(60) Provisional application No. 60/918,198, filed on Mar. 15, 2007.

(51) Int. Cl. A61K 38/22; C07K 9/00; C07K 14/505

(52) U.S. Cl. 514/8; 530/322; 530/397

(57) ABSTRACT

Compositions containing an N-linked oligosaccharide having little or no terminal sialic acid residues which is attached to a therapeutic protein that is bonded to a glycol polymer.

### RELATED APPLICATION INFORMATION

This application claims the benefit of U.S. provisional patent application No. 60/918,198, filed Mar. 15, 2007, the disclosure of which is incorporated in its entirety herein by reference, and is a continuation-in-part of U.S. patent application Ser. No. 11/584,832, filed Oct. 23, 2006, the disclosure of which is incorporated in its entirety herein by reference, which claims the benefit of U.S. provisional patent application No. 60/729,429, filed Oct. 21, 2005, the disclosure of which is incorporated in its entirety herein by reference.

### BACKGROUND OF THE INVENTION

The biologic activity of N-terminally glycosylated therapeutic proteins produced in transgenic avian oviduct tissue (i.e., magnum tissue) or avian oviduct type cells (i.e., tubular gland cells) in culture can be enhanced while the pharmacokinetics are decreased, both apparently the result of little or no sialic acid being present in the N-linked oligosaccharide structures.

Erythropoietin (EPO) stimulates the proliferation and differentiation of erythroid precursor cells to produce mature erythrocytes, a process known as erythropoiesis. This naturally occurring glycoprotein is produced in the kidney and circulates in the blood to stimulate erythropoiesis in the bone marrow. Human derived EPO is heavily glycosylated with one serine (O)-linked oligosaccharide and three asparagine (N)-linked oligosaccharides. The cDNA for EPO has been cloned and expressed in Chinese hamster ovary (CHO) cells and the resulting carbohydrate composition of the CHO-derived EPO has been extensively characterized. The N-linked oligosaccharides of recombinant EPO produced in CHO cells are mostly tetraantennary complex-type with N-acetyllactosamine repeats, similar to natural human urinary EPO. The same is true for the N-linked oligosaccharides on recombinant human EPO obtained from baby hamster kidney cells.

The carbohydrate chains of a glycoprotein may affect its conformation leading to changes in intracellular transport and secretion, solubility and susceptibility to proteases. The carbohydrate chains may also effect in vivo half life by altering routes of clearance and receptor binding properties. Studies to examine the role of EPO glycosylation have used EPO treated with glycosidases, EPO synthesized in the presence of inhibitors of carbohydrate addition and/or processing and EPO expressed in cell mutants with a defect in any of the glycosyltransferases. The removal of sialic acid from native and recombinant CHO derived EPO can increase its in vitro biologic activity, possibly by increasing its affinity to receptors on target cells. However, removal of sialic acid residues destroys the in vivo biologic activity of EPO which correlates with its rapid clearance from the circulation by interaction with hepatic galactosyl receptors. Fractionation of recombinant EPO containing either biantennary and tetraantennary N-linked oligosaccharides demonstrated that EPO containing more extensively branched chains exhibits greater in vivo biologic activity, suggesting a possible unique role for the complex N-linked oligosaccharides in vivo.

It has been elucidated that the in vivo erythropoiesis activity of EPO is dependent on the presence of sialic acid contained on the N-linked EPO oligosaccharide structures. It has been shown that the erythropoietin must be fully glycosylated, including N-linked terminal sialic acids, to accomplish its hormone action in vivo. Asialoerythropoietin (i.e., EPO that had been de-sialated) is rapidly cleared from the circulation by hepatic cells but has an increased in vitro biologic activity possibly due to its increased affinity to receptors on target cells. See, for example, Wasley et al (1991) The importance of N- and O- Linked Oligosaccharides for the Biosynthesis and In Vitro and In Vivo Biologic Activities of Erythropoietin, Blood, vol 77, p 2624-2632; and Jelkmann (2005) Recombinant Erythropoietins—The Role of Glycosylation in Receptor Binding, Action and Degradation, November 2005, European Kidney &. Urological Disease, p 22-24 and Fukuda et al (1989) Survival of Recombinant Erythropoietin in the Circulation: The Role of Carbohydrates, vol 73, p 84-89. The disclosures of Fukuda, Wasley and Jelkmann are incorporated in their entirety herein by reference.

There are a number of previous reports of the PEGylation of erythropoietin. U.S. Pat. No. 6,077,939, the disclosure of which is incorporated in its entirety herein by reference, describes a process for attaching a PEG to the N-terminal alpha carbon of a protein (such as EPO) that has previously been subjected to a transamination reaction. The described conjugates can contain a PEG linked to EPO via a hydrazone, reduced hydrazone, oxime, or reduced oxime linkage.

U.S. Patent Application Publication No. 2002/0081734, the disclosure of which is incorporated in its entirety herein by reference, describes preparing mutant forms of EPO having a cysteine residue introduced at the thirty-eighth position and then PEGylating the mutein at the introduced cysteine residue via a PEG-maleimide derivative.

U.S. Pat. No. 6,753,165, the disclosure of which is incorporated in its entirety herein by reference, describes methods for making soluble proteins (including EPO) having free cysteines. The publication also describes modifying the soluble proteins by attaching a PEG moiety at the free cysteine via a PEG derivative bearing a vinylsulfone, maleimide or iodacetyl moiety.

U.S. Patent Application Publication No. 2003/0191291, the disclosure of which is incorporated in its entirety herein by reference, describes proteins having EPO activity that are prepared using non-recombinant technology. The publication further describes such proteins that are polymer-modified in a defined manner.

U.S. Patent Application Publication No. 2002/0115833, the disclosure of which is incorporated in its entirety herein by reference, describes an EPO glycoprotein covalently linked to one poly(ethylene glycol) group by way of a specific linkage containing an amide bond with the N-terminal alpha-amino group of the EPO glycoprotein.

US Patent Application Publication No. 2006/0182711, the disclosure of which is incorporated in its entirety herein by reference, discloses PEG conjugated to EPO moieties where the EPO moieties have the ability to stimulate red blood cell production.

CERA (continuos erythropoietin receptor activator) is a PEGylated form of erythropoietin produced in CHO cells. Patents that may be related to CERA are U.S. Pat. No. 7,128,913, issued Oct. 31, 2006, the disclosure of which is incorporated in its entirety herein by reference and/or U.S. Pat. No. 6,340,742, issued Jan. 22, 2002, the disclosure of which is incorporated in its entirety herein by reference.

One problem common to each of these pegylated forms of EPO is that though the biological half life of the EPO is extended by the presence of the PEG polymer, the EPO has a reduced activity as a result of the attached polymer, as is well know in the art.

What is needed are new and easily manufactured therapeutic protein compositions such as erythropoietin compositions having both a high level of activity and an extended biological half-life providing for more cost effective and improved treatment of patients.

### SUMMARY OF THE INVENTION

It has been discovered that glycolation (e.g., PEGylation) of transgenic avian or poultry derived erythropoietin can produce an erythropoietin product having a high biologic activity and a lengthened biological half life (PK) resulting in a particularly useful EPO. Transgenic avian or transgenic poultry derived erythropoietin as it is naturally produced in the oviduct contains little or no sialic acid. This trait has been implicated in the increased biologic activity seen in recombinant EPO produced in the bird oviduct compared to recombinant EPO produced in mammalian cells which is fully sialated. See, for example, FIG. 3 which shows the ED₅₀ of the transgenic avian derived EPO to be approximately 7 fold higher than that of EPO produced in CHO cells. This enhanced activity, in combination with glycolation (e.g., pegylation), can provide for a surprisingly effective EPO with regard to pharmacodynamics represented by increase in hematocrit of the subject to which the EPO is administered. Similarly, it is contemplated that therapeutic proteins, in addition to EPO, having an N-linked oligosaccharide produced in the oviduct of an avian, such as chicken or turkey, can be glycolated to yield a therapeutic protein preferable to that of the same pegylated therapeutic produced by pegylating a protein produced using standard cell culture techniques (i.e., mammalian cells in culture).

In one aspect, the invention is directed to a therapeutic protein molecule such as an erythropoietin molecule having an N-linked oligosaccharide wherein the N-linked oligosaccharide does not contain a sialic acid, for example, a terminal sialic acid, and the therapeutic protein is chemically bonded to a glycol polymer. In one embodiment, one N-linked oligosaccharide of an asialic therapeutic protein such as EPO does not contain a sialic acid, for example, a terminal sialic acid. In another embodiment, two N-linked oligosaccharides of the asialic therapeutic protein such as EPO do not contain a sialic acid, for example, a terminal sialic acid. In another embodiment, three N-linked oligosaccharides of the asialic therapeutic protein such as EPO do not contain a sialic acid, for example, a terminal sialic acid. In another embodiment, none of the N-linked oligosaccharides of the asialic therapeutic protein such as EPO contain a sialic acid, for example, a terminal sialic acid. The glycol polymer may be bonded (e.g., covalently bonded) to at least one of an amino group, a hydroxyl group, a sulfhydryl group and a carboxyl group of the therapeutic protein such as EPO. It is contemplated that in addition to covalent bonding the chemical bonding may be ionic bonding, hydrogen bonding and/or Van der Waal's interaction.

The invention also includes methods of producing therapeutic protein molecules such as erythropoietin molecules and compositions containing the therapeutic protein molecules of the invention. For example, the invention includes methods of producing therapeutic protein molecules such as erythropoietin molecules which do not contain sialic acid terminally linked to an oligosaccharide; and covalently bonding a glycol polymer to the therapeutic protein molecule. For example, the invention is directed to producing a composition containing a therapeutic protein such as erythropoietin obtained from a transgenic avian, for example, a transgenic chicken, chemically (e.g., covalently) bonded to a glycol polymer.

In one embodiment, the therapeutic protein molecules such as erythropoietin molecules of the invention contain an O-linked oligosaccharide. In one embodiment, the therapeutic protein molecules of the invention contain an O-linked oligosaccharide and an N-linked oligosaccharide.

In one embodiment, the asialic therapeutic protein molecules such as the asialic erythropoietin molecules are exogenous to avians, for example, exogenous to a chicken, turkey or quail. In one particularly useful embodiment, the therapeutic protein molecule are human protein molecules. For example, the therapeutic protein molecule may have the amino acid sequence set forth in FIG. 2B.

In one embodiment, the asialic therapeutic protein such as asialic erythropoietin is produced in an oviduct cell of the transgenic avian, for example, in a tubular gland cell of the avian.

The invention also includes pharmaceutical compositions comprising a glycolated asalic therapeutic protein of the invention. In one aspect, the invention is directed to methods of treatment comprising administering to a patient in need thereof glycolated asalic therapeutic protein of the invention such as EPO. In one aspect, the invention is directed to methods of treatment comprising administering to a patient in need thereof a pharmaceutical composition containing a glycolated asalic therapeutic protein of the invention such as EPO.

The invention also includes methods of increasing the half-life of an asialic therapeutic protein such as EPO in a patient comprising bonding a glycol polymer to the asialic protein. The invention is also directed to methods of increasing the red blood cell count in a patient by administering to a patient a glycolated asialic EPO of the invention.

The invention contemplates the application of any useful glycol polymer for attachment to a protein, for example, asialic EPO. For example, the glycol polymer may be a polyalkylene glycol such as a polyethylene glycol and a polypropylene glycol. The invention is not limited to glycol polymers of any particular molecular weight. For example, the glycol polymers may have a molecular weight of about 200 to about 400,000, for example, a molecular weight of about 200 to about 20,000. In another example, the glycol polymers may have a molecular weight of about 300 to about 200,000, for example, a molecular weight of about 2,000 to about 60,000

In a particularly useful embodiment of the invention poultry derived EPO is linked to the PEG molecule shown as Structure I below.

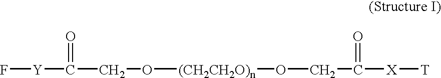

wherein: X and Y are linking groups selected from NH and O;
T is a saccharide or a saccharide derivative; and
F is an activated group, which can react with amino, hydroxyl, carboxyl or thiol groups of the proteins in an aqueous phase without enzymes or other catalysts, as is well know in the art.
For example, transgenic poultry derived therapeutic proteins such as EPO are linked to the PEG of Structure VII which is shown below. This PEG is particularly effective when coupled to avian derived EPO. For example, the PEG-EPO yields an unexpectedly high PD as can be seen in FIG. 6.

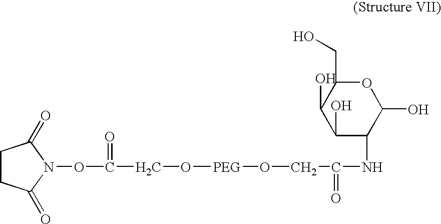

As is understood in the art, coupling of the PEG of Structure VII to a protein such as EPO can produce a PEGylated protein of Structure VIII. PEG in Structure VII and VIII represents (CH₂CH₂O)ₙ and Protein N in VIII represents the protein such as EPO and an amino group.

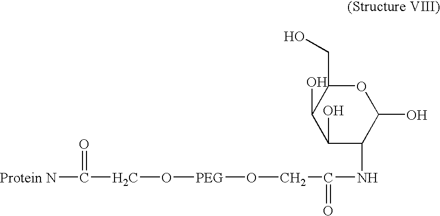

The invention contemplates the linking of the glycol polymer to the protein by any useful chemical bonding methods known in the art. In one embodiment, the glycol polymer is covalently bonded to an amino group of the amino acid sequence. In another example, the glycol polymer is covalently bonded to a carboxyl group of the amino acid sequence.

In one specific embodiment, the PEG polymer was bound to the poultry derived EPO of the invention as follows. Three parts of 100 mM sodium borate buffer, pH of 9.3, was added to one part of a stock solution of 0.2 to 0.4 mg/ml TPD EPO contained in 10 mM phosphate buffer, pH 6.0. The 35 KDa Glucose PEG (Structure VII) was dissolved in acetonitrile to a concentration of about 20 mg/ml. The PEG solution was slowly added to the EPO solution with stirring until a final EPO:PEG ratio of 10:1 was reached. The reaction mixture was stirred for an additional 60 minutes at room temperature to complete the conjugation reaction. The invention contemplates the employment of any useful purification technique. For example, size exclusion chromatography has can be useful for purification of PEG-EPO produced in accordance with the invention.

In one useful embodiment, the therapeutic amino acid sequence obtained from a transgenic avian is a glycoprotein and comprises a glycol polymer covalently bonded to an oligosaccharide of the therapeutic amino acid sequence. The invention contemplates the linking of the glycol polymer to any component of the glycosylation of the therapeutic amino acid sequence. For example, and without limitation, the invention contemplates the linking of the glycol polymer to n-acetyl-galactosamine, n-acetyl-glucosamine, galactose and/or any other carbohydrate structure which may be present in the glycosylation.

In one useful aspect, the invention is drawn to compositions which contain a glycosylated therapeutic amino acid sequence obtained from a transgenic avian, such as a transgenic chicken, wherein the therapeutic amino acid sequence is a glycoprotein associated with a glycol polymer. For example, the glycoprotein may be associated with the glycol polymer by a chemical interaction such as ionic bonding or hydrogen bonding. In one particularly useful embodiment, the glycoprotein is covalently bonded to the glycol polymer. In a particularly useful embodiment of the invention, the therapeutic amino acid sequence is an exogenous amino acid sequence. For example, the therapeutic amino acid sequence may be an amino acid sequence endogenous to a human, such as human EPO.

In addition to EPO, it is contemplated that the biologic activity of other N-terminally glycosylated therapeutic proteins, in particular, those produced in transgenic avian oviduct tissue such as magnum tissue or avian oviduct type cells in culture such as cell lines derived from tubular gland cells, is enhanced and biological half life decreased, each because there is little or no sialic acid present in the N-linked oligosaccharide structure and as such glycolation (e.g., PEGylation) of such protein can yield a particularly useful therapeutic product. Such therapeutic proteins which can contain an N-linked oligosaccharide include without limitation: Factor VIII, Factor VIIa, Factor IX, alteplase tPA, Reteplase tPA (differs from h tPA—3 of 5 domains deleted), growth hormone (e.g., hGH), thyroid stimulatin hormone (TSH), follicle stimulating hormone (FSH), follitropin-beta, calcitonin, platelet derived growth factor (PDGF), keratinocyte growth factor, insulin-like growth factor-1 (IGF-1), IGFBP-3, GM-CSF, inteferons such as INF-beta, e.g., INF-beta1b and IFN-gamma, e.g., IFN-gamma 1b, interleukins such as IL-3 and IL-12, TNFR-IgG fragment fusion protein, beta glucocerebrosidase, asparaginase, urokinase, adenosin deaminase, agalsidase alfa, idursulfase, alpha-L-iduronidase, galsulfase: arylsulfatase, galsulfase: arylsulfatase B, BM 102, N-acetylgalactosamine-4-sulfatase, hASB, activated protein C, domase-alpha DNAse, anakinra, eptotermin alfa, Protein C, dibotermin alfa, for example, the human forms of each of these proteins. The invention also includes the PEGylated forms of each of these avian derived proteins, methods of PEGylating each of these avian derived proteins and methods of using each of these PEGylated avian derived proteins, each in accordance with the invention.

In one aspect of the invention, the asialic glycosylation is provided by an oviduct cell of the transgenic avian or an avian oviduct cell in culture or a cell in culture derived at least partially from an avian oviduct cell such as a tubular gland cell. In one particularly useful embodiment, the oviduct cell can be a tubular gland cell. See, for example, U.S. patent application Ser. No. 11/454,399, filed Jun. 16, 2006, the disclosure of which is incorporated in its entirety herein by reference.

In one embodiment, the invention is drawn to oligosaccharide structures being linked to the proteins by linkages provided for in an avian gene expression system. For example, the therapeutic amino acid sequence may be O-glycosylated and/or the therapeutic amino acid sequence may be N-glycosylated.

In one embodiment, therapeutic proteins produced in accordance with the present invention are soluble in an aqueous phase or are substantially soluble in an aqueous phase. The proteins produced in accordance with the present invention can be nonimmunogenic or have reduce immunogenicity relative to an otherwise identical protein that is not glycolated.

Throughout this specification specific examples and specific references are directed to EPO and it is understood that such specific examples and specific references are contemplated as being applicable to other therapeutic proteins, for example, other therapeutic proteins having an N-linked oligosaccharide, for example, therapeutic proteins produced in transgenic avians such as chickens.

Any useful combination of features described herein is included within the scope of the present invention provided that the features included in any such combination are not mutually inconsistent as will be apparent from the context, this specification, and the knowledge of one of ordinary skill in the art.

Additional objects and aspects of the present invention will become more apparent upon review of the detailed description set forth below when taken in conjunction with the accompanying figures, which are briefly described as follows.

### BRIEF DESCRIPTION OF THE DRAWINGS

FIGS. 1A and 1B shows representative deduced N-linked oligosaccharide structures for TPD EPO produced essentially as disclosed in U.S. patent application Ser. No. 11/973,853, filed Oct. 11, 2007, the disclosure of which is incorporated in its entirety herein by reference.

FIG. 2A shows the human EPO nucleotide coding sequence used to produce TPD EPO employed herein. FIG. 2B shows the mature EPO amino acid sequence produced from the coding sequence shown in FIG. 2A.

FIG. 3 shows the in vitro activity of poultry derived EPO (□) compared to mammalian cell derived EPO (∘).

FIG. 4 shows the PK measurements for a 5 KDa PEGylated TPD EPO (♦) and a 20 KDa PEGylated TPD EPO (▪), described in Examples 2 and 3 respectively, compared to non-PEGylated TPD EPO (X) and fully sialated EPO produced in CHO cells (▴). The injections were performed subcutaneously which accounts for the initial rise in EPO detected at 4 h.

FIG. 5 shows the PK measurements for the 40 KDa PEGylated TPD EPO (▪) and the 35 KDa PEGylated TPD EPO (♦) described in Examples 4 and 5 respectively, compared to non-PEGylated TPD EPO (X) and fully sialated EPO produced in CHO cells (▴). As in FIG. 4, the injections were performed subcutaneously which accounts for the initial rise in EPO detected at 4 h.

FIG. 6 shows the hematocrit measurements for the 35 KDa PEGylated TPD EPO (TPD EPO) (▴) and the 40 KDa PEGylated TPD EPO (TPD EPO) (♦), as described in Example 6, compared to fully sialated EPO produced in CHO cells (X).

### DEFINITIONS AND ABBREVIATIONS

Certain definitions are set forth herein to illustrate and define the meaning and scope of the various terms used to describe the invention herein.

The term “avian” as used herein refers to any species, subspecies or race of organism of the taxonomic class ava, such as, but not limited to, chicken, turkey, duck, goose, quail, pheasants, parrots, finches, hawks, crows and ratites including ostrich, emu and cassowary. The term includes the various known strains of Gallus gallus, or chickens, (for example, White Leghorn, Brown Leghorn, Barred-Rock, Sussex, New Hampshire, Rhode Island, Australorp, Minorca, Amrox, California Gray), as well as strains of turkeys, pheasants, quails, duck, ostriches and other poultry commonly bred in commercial quantities. It also includes an individual avian organism in all stages of development, including embryonic and fetal stages. The term “avian” also may denote “pertaining to a bird”, such as “an avian (bird) cell.”

“Asialic” refers to a therapeutic protein such as erythropoietin that does not contain sufficient sialic acid residues (i.e., N-linked oligosaccharide sialic acid residues) to provide for a therapeutically useful biological half-life of the protein. For example, following administration by injection, more than 95% of 200 IU (16.7 μg) of asialated EPO will clear from the body of a rat of approximately 250 grams within 4 h or 8 h or 12 h or 24 of injection. Asialaic EPO typically, though not exclusively, contains three N-linked oligosaccharide structures. In one useful embodiment, asialic EPO is produced by heterologous gene expression of an EPO encoding nucleotide sequence in the magnum tissue of a transgenic bird such as a chicken.

The terms “heterologous”, “exogenous” and “foreign” are used interchangeably herein and in general refer to a biomolecule such as a nucleic acid or a protein that is not normally found in a particular cell, tissue or other component contained in or produced by an organism.

“Conjugation buffer” is 7.5 mg/ml NaCl, 1.8 mg/ml dibasic sodium phosphate, 1.3 mg/ml monobasic sodium phosphate, 0.1 mg/ml edetate disodium, 0.4 mg/ml Tween 80, pH adjusted to 7.0.

The term “cytokine” as used herein refers to a proteinaceous signalling compound involved in inter-cell communication. Cytokines play a major role in a variety of immunological, inflammatory and infectious diseases. They are also involved in several developmental processes during embryogenesis. Cytokines are produced by a wide variety of cell types, both haemopoietic and non-haemopoietic, and can have effects on nearby cells or cells throughout the organism, sometimes strongly dependent on the presence of other chemicals and cytokines. Cytokines are typically smaller water-soluble proteins, for example, glycoproteins, with a mass of 8-30 kDa.

“Excludes” mean does not contain or does not include.

“Glycolation” refers to the addition of a glycol polymer to a molecule such as the addition of a glycol polymer to a protein therapeutic. “Glycolated” refers to a substance, such as a protein therapeutic, to which a glycol polymer has been added.

A “glycol polymer” as used herein refers to any useful alkene, alkane or alkyne (and combinations thereof) polymer glycol. Examples include, without limitation, polypropylene glycol, polyethylene glycol and polybutylene glycol.

The term “PEG” means polyethylene glycol.

As used herein a “standard protein therapeutic” is a protein therapeutic that does not contain a poultry derived glycosylation pattern and a glycol polymer. A standard protein therapeutic can be a protein therapeutic containing a poultry derived glycosylation pattern or a glycol polymer.

The term, “substantially” means mostly, for example, more than 50%. For example, a protein substantially lacking sialic acid does not have sialic acid attached to at least half of the sites where sialic acid would ordinarily be expected to be attached. For example, an N-linked oligosaccharide substantially excludes or is substantially lacking sialic acid if more than half of the terminal galactose residues on the oligosaccharide do not have an attached sialic acid.

“Terminal sialic acid” and terminal “sialic acid residue” refer to a sialic acid attached to the end of an oligosaccharide chain which is attached to a therapeutic protein. Typically, a terminal sialic acid is attached to a galactose molecule.

“Therapeutic protein”, “protein therapeutic”, “pharmaceutical protein” “therapeutic amino acid sequence” each refer to an amino acid sequence which in whole or in part makes up a drug. A pharmaceutical composition or therapeutic composition is a liquid formulation that includes one or more protein therapeutics, pharmaceutical proteins, therapeutic amino acid sequences or therapeutic proteins.

As used herein, a “transgenic avian” is any avian, as defined herein, in which one or more of the cells of the avian contain heterologous nucleic acid introduced by manipulation, such as by transgenic techniques. The nucleic acid may be introduced into a cell, directly or indirectly, by introduction into a precursor of the cell by way of deliberate genetic manipulation, for example and without limitation, by microinjection or by infection with a recombinant retrovirus, for example, injection of a recombinant replication deficient retrovirus into the subgerminal cavity of an avian embryo. The heterologous nucleic acid may be an artificial chromosome or may be integrated within a chromosome of the avian, or it may be extrachromosomally replicating DNA.

As used herein, “treating” or “treating a condition” refers to administering a pharmaceutical composition for preventing disease and/or treating disease. To prevent disease refers to prophylactic treatment of a patient who is not yet ill, but who is susceptible to, or otherwise at risk of, contracting a particular disease. To “treat disease” or “use for therapeutic treatment” refers to administering treatment to a patient already suffering from a disease to ameliorate the disease and improve the patient's well being. Thus, “treating” or “treating a condition” is the administration to a subject one or more therapeutic proteins either for therapeutic or prophylactic purposes.

“Linear” in reference to the geometry, architecture or overall structure of a polymer, refers to polymer single monomer derived backbone.

“Branched,” in reference to the geometry, architecture or overall structure of a polymer, refers to polymer having 2 or more polymer “arms” extending from a single group, such as an L group that may be derived from an initiator employed in an atom transfer radical polymerization reaction. A branched polymer may possess 2 polymer arms, 3 polymer arms, 4 polymer arms, 5 polymer arms, 6 polymer arms, 8 polymer arms or more. For the purpose of this disclosure, compounds having three or more polymer arms extending from a single linear group are denoted as having a “comb” structure or “comb” architecture.

“Pharmaceutically acceptable” composition or “pharmaceutical composition” refers to a composition comprising a compound of the invention and a pharmaceutically acceptable excipient or pharmaceutically acceptable excipients.

The term “salt” includes, without limitation, acid addition salts including hydrochlorides, hydrobromides, phosphates, sulphates, hydrogen sulphates, alkylsulphonates, arylsulphonates, acetates, benzoates, citrates, maleates, fumarates, succinates, lactates, and tartrates; salts of alkali metal cations such as Na⁺, K⁺, Li⁺ (e.g., NaCl, KCl) organic amine salts or alkali earth metal salts such as Mg or Ca salts.

“Pharmaceutically acceptable excipient” and “pharmaceutically acceptable carrier” refer to an excipient that can be included in the compositions of the invention and that causes no significant adverse toxicological effect on the patient. Non-limiting examples of pharmaceutically acceptable excipients include water, NaCl, normal saline solutions, lactated Ringer's, normal sucrose, normal glucose and the like.

“Patient” or “subject in need thereof” refers to a living organism suffering from or prone to a condition that can be prevented or treated by administering a therapeutic protein of the invention or a pharmaceutical composition containing a therapeutic protein of the invention as provided herein. Non-limiting examples of patients include humans and other mammals and non-mammalian animals.

The term “poultry” means domesticated birds used for eggs or meat such as chickens and turkeys.

“Therapeutically effective amount” refers to an amount of a conjugated biologically active agent or of a pharmaceutical composition useful for treating, ameliorating, or preventing an identified disease or condition, or for exhibiting a detectable therapeutic or inhibitory effect. The effect can be detected by any assay method known in the art.

The “biological half life” or “biologic half life” of a substance is the pharmacokinetic parameter which specifies the time required for one half of the substance to be removed from an organism following introduction of the substance into the organism.

“Reactive group” refers to a functional group that is capable of forming a covalent linkage consisting of one or more bonds to a biologically active agent.

The abbreviation “g” means grams. The abbreviation “ml” means milliliters. The abbreviation “mg” means milligrams. The abbreviation “PEG” means polyethylene glycol. The abbreviation “KDa” means kilodalton. “° C.” means degrees centigrade. The abbreviation “mM” means millimolar. The abbreviation “h” means hours. The abbreviation “mU” means milliunits. The abbreviation “IU” means international units. The abbreviation “μl” means microliters. The abbreviation “PK” means pharmacokinetics. The abbreviation “PD” means pharmacodynamics.

### DETAILED DESCRIPTION

The biologic activity of N-terminally glycosylated therapeutic proteins produced in transgenic avian oviduct tissue (i.e., magnum tissue) or avian oviduct type cells (i.e., tubular gland cells) in culture is enhanced while the pharmacokinetics is decreased since little or no sialic acid is present in the N-linked oligosaccharide structure. Glycolation (e.g., PEGylation) of such protein can yield a particularly useful therapeutic product.

A particularly useful aspect of the invention is that glycosylated therapeutic proteins, in particular, human proteins, produced in transgenic avian oviduct tissue (e.g., tubular gland cells) are naturally asialic. The proteins can be easily produced in large quantities in a transgenic avian system. In addition to containing little or no sialic acid, other aspects of the N-linked oligosaccharides of proteins produced in transgenic avians are not shared with oligosaccharides of the same protein produced in mammalian cells. Such differences are readily apparent based upon the knowledge of a practitioner of skill in the art in conjunction with the disclosure of the specification.

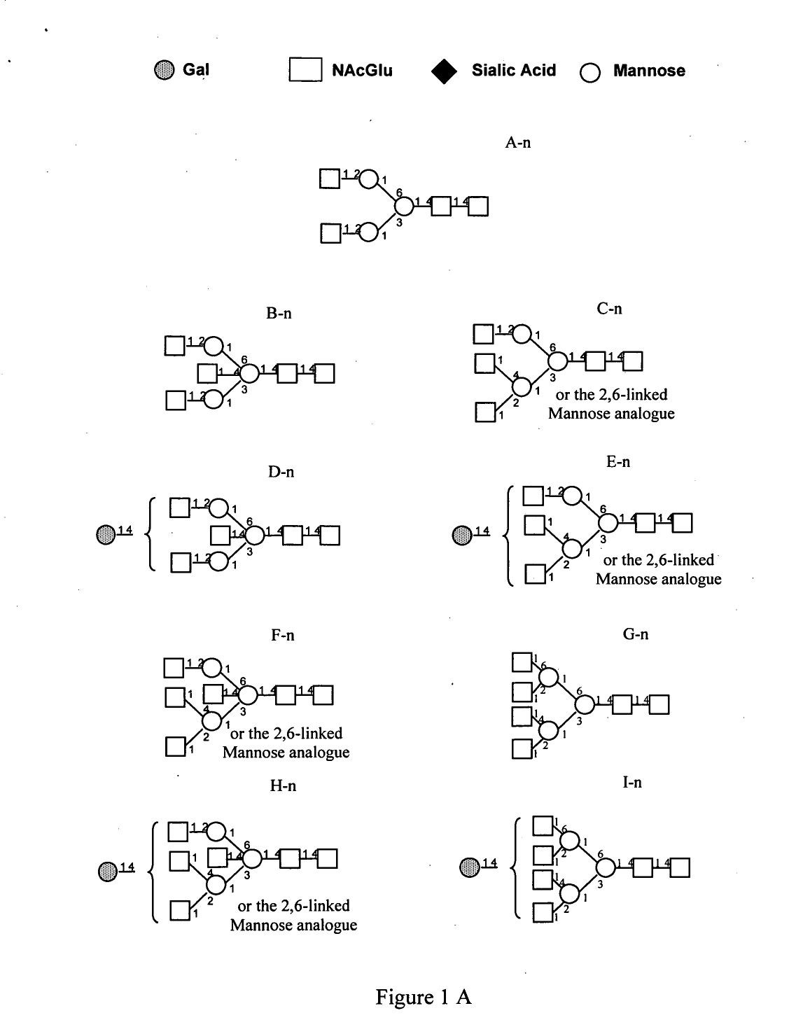

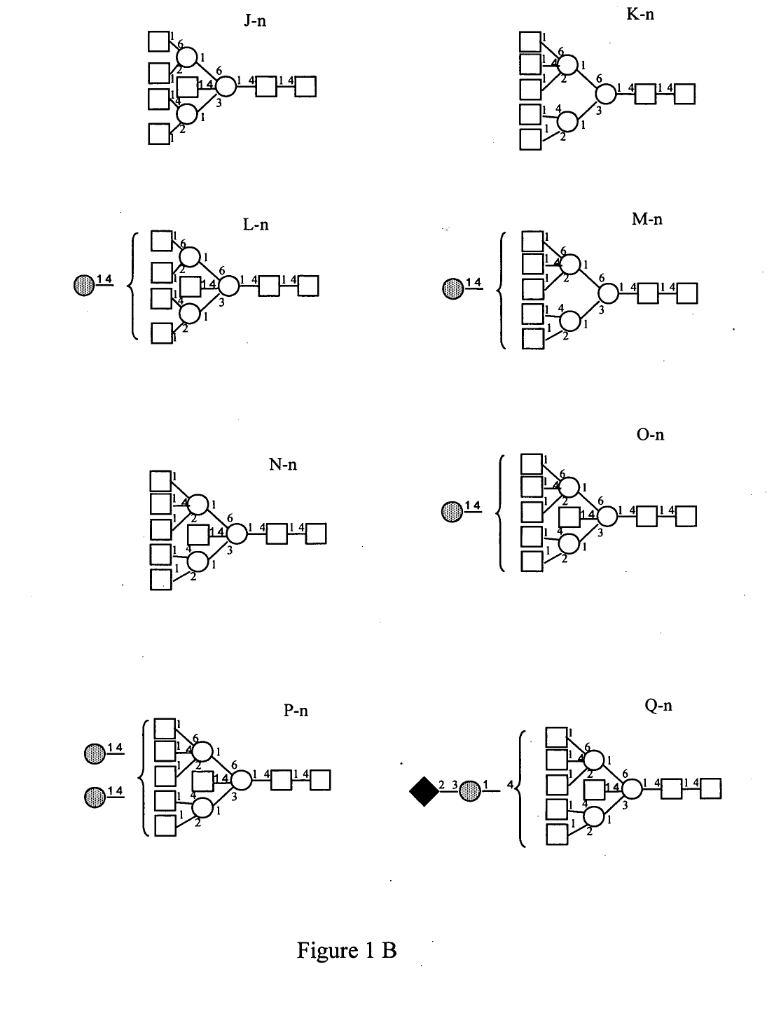

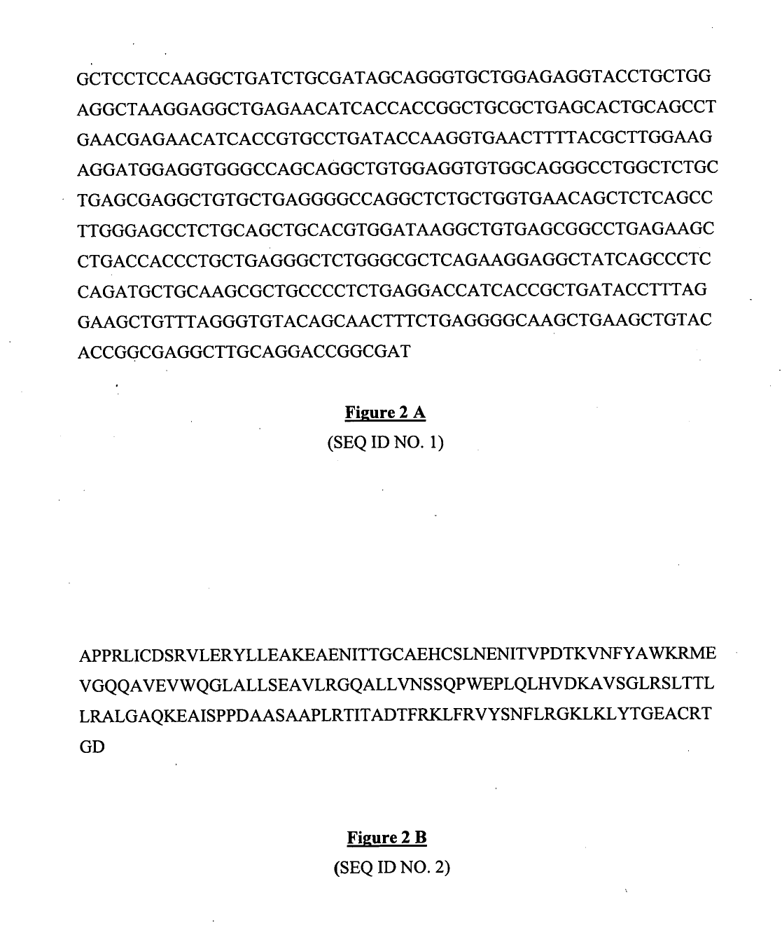

This invention contemplates the bonding of a water soluble polymer to an asialic therapeutic protein such as EPO. In a particularly useful embodiment, the asialic therapeutic proteins are produced in the oviduct of transgenic birds including transgenic poultry such as transgenic chicken, turkey and quail. For example, the invention specifically contemplates the glycolation, for example, PEGylation, of asialic EPO (e.g., asialic human EPO). Asialic EPO molecules may be produced by avians, including without limitation, chicken, turkey, duck, goose, quail, pheasants, parrots, finches, hawks, crows and ratites including ostrich, emu and cassowary. For example, FIG. 1 shows some of the identified N-linked oligosaccharide structures of human EPO (EPO shown in FIG. 2B) that were produced in chickens essentially as disclosed in U.S. Pat. No. 6,730,822, issued May 4, 2004, the disclosure of which is incorporated in its entirety herein by reference. It can be seen that only one of these structures which has been elucidated may have a single terminal sialic acid residue attached. In one particular embodiment, the invention is drawn to glycolation of this asialic EPO protein produced in chickens.

One glycol polymer that is particularly useful in accordance with the present invention is polyethylene glycol (PEG). PEG is a hydrophilic, biocompatible and non-toxic polymer of general formula H (OCH₂CH₂)nOH, wherein n>4. Its molecular weight can vary substantially, for example, from 200 to 20,000 Dalton. The invention is not specifically drawn to any particular method of attaching PEG molecules to proteins or any particular molecular weight of PEG employed.

Many useful methods of glycolating proteins are known in the art and the present invention contemplates the employment of each such method. For example, the invention contemplates any useful method of PEGylation to produce therapeutic proteins such as PEGylated asialic EPO as disclosed herein. In one example, certain well known methods for PEGylating proteins use compounds such as N-hydroxysuccinimide (NHS)-PEG to attach PEG to free amines, typically at lysine residues or at the N-terminal amino acid.

Site specific methods of PEGylation are also included in the present invention. One such method attaches PEG to cysteine residues using cysteine-reactive PEGs. A number of highly specific, cysteine-reactive PEGs with different reactive groups (e.g., maleimide, vinylsulfone) and different size PEGs (e.g., 2-40 kDa) are commercially available. At neutral pH, these PEG reagents selectively attach to “free” cysteine residues, i.e., cysteine residues not involved in disulfide bonds. Through in vitro mutagenesis using recombinant DNA techniques, additional cysteine residues can be introduced at any useful position in the therapeutic protein. The newly added “free” cysteines can serve as sites for the specific attachment of a PEG molecule using cysteine-reactive PEGs. The added cysteine residue can be a substitution for an existing amino acid in a protein, added preceding the amino-terminus of the protein or after the carboxy-terminus of the protein, or inserted between two amino acids in the protein. Alternatively, one of two cysteines involved in a native disulfide bond, which may be present in certain therapeutic proteins, may be deleted or substituted with another amino acid, leaving a native cysteine (i.e., the cysteine residue in the protein that normally would form a disulfide bond with the deleted or substituted cysteine residue) free and available for chemical modification. In one embodiment, the amino acid substituted for the cysteine would be a neutral amino acid such as serine or alanine. In addition, disulfide bonds can be reduced and alkylated with iodoacetimide without impairing biological activity providing targets for deletion or substitution by another amino acid. In one embodiment, a cysteine residue is incorporated into the amino acid sequence of a modified TPD EPO protein, employing methods known in the art, for example, recombinant DNA methodologies.

In one embodiment, methods for preparing a glycolated, for example, PEGylated glycoprotein comprise the steps of (a) reacting the protein with polyethylene glycol, such as a reactive ester or aldehyde derivative of PEG, under conditions whereby the protein becomes attached to one or more PEG groups and (b) obtaining the reaction product(s). In general, the optimal reaction conditions for the reactions will be determined case by case based using known parameters for the desired result.

There are a number of attachment methods available to those skilled in the art. See, for example, EP 0 401 384, the disclosure of which is hereby incorporated by reference; see also, Malik et al. (1992), Exp. Hematol., 20:1028-1035; Francis (1992), Focus on Growth Factors, 3(2):4-10, (published by Mediscript, Mountain Court, Friern Barnet Lane, London N20 OLD, UK); EP 0 154 316; EP 0 401 384; WO 92/16221; WO 95/34326; and the other publications cited herein that relate to addition of a glycol polymer to a protein (e.g., PEGylation). The disclosure of each of the references cited herein, including patent applications, issued patents and journal articles, is hereby incorporated by reference.

US Patent Application Publication No. 2006/0182711, published Aug. 17, 2006, the disclosure of which is incorporated in its entirety herein by reference, has extensive disclosure regarding compositions and methods for the attachment of water soluble polymers to EPO and as such the disclosure of Application Publication No. 2006/0182711 is contemplated for making and using the present invention.

In one embodiment, glycol polymer molecules such as polyethylene glycol polymer molecules can be “activated” to facilitate coupling of the glycol polymer molecule to the protein in accordance with the invention. Examples of preparation of such activated glycol polymers are provided in the following references which are hereby incorporated by reference: K. Yoshinaga and J. M. Harris, J. Bioact. Comp. Polym., 1, 17-24 (1989); K. Nilsson and K. Mosbach, Methods in Enzymology, 104, 56 (1984); C. Delgado, G. E. Francis, and D. Fisher, in “Separations Using Aqueous Phase Systems,” D. Fisher and I. A. Sutherland, Eds., Plenum, London, 1989, pp. 211-213; M.-B. Stark and J. K. Holmberg, Biotech. Bioeng., 34, 942 (1989); J. M. Harris and K. Yoshinaga, J. Bioact. Compat. Polym., 4, 281 (1989); H. Walter, D. E. Brooks, and D. Fisher (Editors), “Partitioning in Aqueous Two-Phase Systems,” Academic Press, Orlando, Fla., 1985; D. Fisher and I. A. Sutherland (Editors), “Separations Using Aqueous Phase Systems: Applications in Cell Biology and Biotechnology,” Plenum, London, 1989.

U.S. Pat. No. 4,002,531, issued Jan. 11, 1977, the disclosure of which is incorporated in its entirety herein by reference, describes preparation of PEG acetaldehyde for attaching PEG to protein. Such methods are contemplated for the attachment of PEG to protein (e.g., asialic EPO) in accordance with the present invention.

The invention contemplates the application of any useful glycol polymer to produce glycolated protein therapeutics. The following polymer types are examples of PEG polymers that can be linked to therapeutic proteins disclosed herein such as EPO.

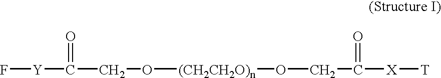

wherein: X and Y are linking groups selected from NH and O;
T is a targeting agent group of a saccharide or its derivatives; and
F is an activated group, which can react with amino, hydroxyl, carboxyl or thiol groups of the proteins in an aqueous phase without enzymes or other catalysts, as is well know in the art. See, for example, Structure VII.

Accordingly, in one embodiment, a composition of the invention comprises a erythropoietin molecule comprising an N-linked oligosaccharide wherein the N-linked oligosaccharide substantially excludes sialic acid and the erythropoietin is chemically bonded to a glycol polymer comprising the structure:

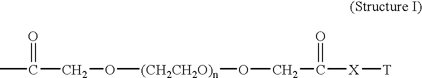

wherein: X is a linking group selected from NH and O; and
T is a saccharide or a saccharide derivative.

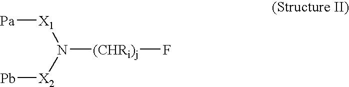

Wherein Pₐ and P_{b} are selected from the group consisting of polyethylene glycol, polypropylene glycol, which are the same or different;
   j is an integer from 1 to 12;
   Rᵢ is selected from the group consisting of H, a C₁₋₁₂ substituted or unsubstituted alkyl, a substituted aryl, an aralkyl, and a heteroalkyl;
   X₁ and X₂ independently are linking groups, wherein X₁ is (CH₂)ₙ, and X₂ is selected from the group consisting of (CH₂)ₙ, (CH₂)ₙOCO, (CH₂)ₙNHCO and (CH₂)ₙCO, wherein n is an integer of from 1-10; and
   F is a functional group capable of reacting with an amino, hydroxyl, carboxyl or thiol groups of the proteins in an aqueous phase without enzymes or other catalysts, as is well know in the art.

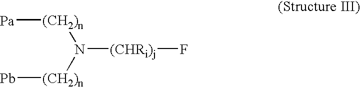

Wherein Pₐ and P_{b} are glycols, which are the same or different. For example, Pₐ and P_{b} can be polyethylene glycol;
and j are independently an integer from 1 to 12;
Ri is selected from the group consisting of H, a C₁₋₁₂ substituted or unsubstituted alkyl, a substituted aryl, an aralkyl, and a heteroalkyl; and
F can be a functional group capable of reacting with an amino group, a hydroxyl group or a thiol group of the protein.

Structures I, II and III and other useful structures and methods which may be employed in the present invention are disclosed in US patent publication No. 2006/0014666, published Jan. 19, 2006, and US patent publication No. 2005/0180946, published Aug. 18, 2005, the disclosures of which are incorporated in their entirety herein by reference. It is noted that the PEG structure shown in Example 4 of publication No. 2006/0014666 is a particularly useful PEG molecule in accordance with the invention. In one embodiment, the PEG structure shown in Example 4 of publication No. 2006/0014666 is a particularly useful PEG to coupled to poultry derived therapeutic proteins of the invention, including, without limitation, poultry derived therapeutic proteins having an N-linked oligosaccharide.

Other useful PEG molecules which can be employed in the present invention include those with a methacrylate backbone. For example, “comb shaped” PEG polymers such as PolyPeg® manufactured by Warwick Effect Polymers, Inc may be used in accordance with the present invention. See, for example, Structure IV below and WO 2004/113394 A2, the disclosure of which is incorporated in its entirety herein by reference.

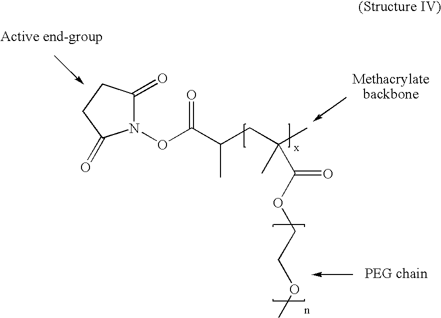

Other useful methods for PEGylating asialic therapeutic proteins such as asialic EPO are disclosed in U.S. Pat. No. 6,586,398, issued Jul. 1, 2003, the disclosure of which is incorporated in its entirety herein by reference.

U.S. Pat. No. 4,179,337, issued Dec. 18, 1979, the disclosure of which is incorporated in its entirety herein by reference, discloses certain methods for attaching PEG to proteins to provide soluble PEG-protein conjugates. Such methods are contemplated for the attachment of PEG to therapeutic proteins in accordance with the present invention.

Glycolation such as PEGylation may be carried out by, for example, an acylation reaction or an alkylation reaction with a reactive or activated polyethylene glycol polymer molecule. Thus, protein products produced according to the present invention include PEGylated proteins wherein the PEG group(s) is (are) attached by acyl or alkyl groups. Such products may be mono-PEGylated or poly-PEGylated (e.g., containing 2-6, and/or 2-5, PEG groups). The PEG groups can be attached to the protein at the alpha or epsilon amino groups of amino acids, but it is also contemplated that the PEG groups could be attached to any group of the protein which is sufficiently reactive to become attached to a PEG group under suitable reaction conditions.

Glycolation such as PEGylation by acylation generally can involve reacting an active ester derivative of glycol polymer such as PEG with the protein. For the acylation reactions, the polymer(s) selected can have a single reactive ester group. Any known or subsequently discovered reactive PEG molecule may be used to carry out the PEGylation reaction. A useful activated PEG ester is PEG esterified to N-hydroxysuccinimide (NHS). As used herein, “acylation” is contemplated to include, without limitation, the following types of linkages between the protein and a glycol polymer such as PEG: amide, carbamate, urethane, and the like (Chamow (1994), Bioconjugate Chem., 5 (2): 133-140). Reaction conditions may be selected from any of those known in the PEGylation art or those subsequently developed, but should avoid conditions such as temperature, solvent and pH that would inactivate the protein (e.g., asialic EPO) to be modified.

Glycolation by acylation can result in a poly-PEGylated protein. In one embodiment, the connecting linkage is an amide. Also, the resulting product may be substantially only (e.g., >95%) mono, di- or tri-PEGylated. However, some species with higher degrees of PEGylation may be formed in amounts depending on the specific reaction conditions used. If desired, certain PEGylated species (e.g., mono, di- or tri-PEGylated) may be separated from the mixture by standard purification techniques, including among others, dialysis, salting-out, ultrafiltration, ion-exchange chromatography, gel filtration chromatography and electrophoresis. In one particularly useful embodiment, a substantially pure mono-PEGylated form of the protein is purified in accordance with the invention.

Glycolation in accordance with the invention, such as PEGylation by alkylation can involve reacting a terminal aldehyde derivative of a glycol polymer such as PEG with the protein in the presence of a reducing agent. For the reductive alkylation reaction, the polymer(s) selected can have a single reactive aldehyde group. An exemplary reactive PEG aldehyde is polyethylene glycol propionaldehyde, which is, water stable, or mono C1-C10 alkoxy or aryloxy derivatives thereof. See, for example, U.S. Pat. No. 5,252,714, issued Oct. 12, 1993, the disclosure of which is incorporated in its entirety herein by reference.

Though glycolation such as PEGylation by alkylation can result in poly-PEGylated protein, one can manipulate the reaction conditions to substantially favor glycolation only at the alpha amino group of the N-terminus of the protein to provide a mono-PEGylated protein, as is understood in the art. In either case the glycol polymer groups are often attached to the protein by a—CH₂—NH-group.

Reductive alkylation to produce a substantially homogeneous population of mono-polymer/protein product can include the steps of:

(a) reacting an asialic therapeutic protein (such as a glycosylated, asialic EPO) with a reactive PEG molecule under reductive alkylation conditions, at a pH suitable to permit selective modification of the alpha amino group at the amino terminus of the protein; and
(b) obtaining the reaction product(s).

The reaction can be performed at a pH which allows one to take advantage of the pKa differences between the epsilon amino groups of the lysine residues and that of the alpha amino group of the N-terminal residue of the protein. In general, if the pH is lower, a larger excess of polymer to protein will be desired (i.e., the less reactive the N-terminal alpha amino group, the more polymer needed to achieve optimal conditions). If the pH is higher, the polymer:protein ratio need not be as large (i.e., more reactive groups are available, so fewer polymer molecules are needed). In one embodiment, the pH can fall within the range of 3 to 9, for example, 3 to 6. For the reductive alkylation, the reducing agent should be stable in aqueous solution and preferably be able to reduce only the Schiff base formed in the initial process of reductive alkylation. Suitable reducing agents may be selected from sodium borohydride, sodium cyanoborohydride, dimethylamine borane, trimethylamine borane and pyridine borane. A particularly suitable reducing agent is sodium cyanoborohydride. Other reaction parameters such as solvent, reaction times, temperatures and means of purification of products can be determined on a case-by-case basis, based on the published information relating to derivatization of proteins with water soluble polymers.

By such selective derivatization, attachment of a glycol polymer that contains a reactive group such as an aldehyde to a protein can be controlled. The conjugation with the polymer takes place predominantly at the N-terminus of the protein without significant modification of other reactive groups, such as the lysine side chain amino groups, occurs. The preparation can typically be greater than 90% monopolymer/protein conjugate, or greater than 95% monopolymer/protein conjugate, with the remainder of observable molecules being unreacted (i.e., protein lacking the polymer moiety).

Glycolation also may be carried out by water soluble polymers having at least one reactive hydroxy group (e.g. polyethylene glycol) that can be reacted with a reagent having a reactive carbonyl, nitrile or sulfone group to convert the hydroxyl group into a reactive Michael acceptor, thereby forming an activated linker useful in modifying various proteins to provide improved biologically-active conjugates. Reactive carbonyl, nitrile or sulfone means a carbonyl, nitrile or sulfone group to which a two carbon group is bonded having a reactive site for thiol-specific coupling on the second carbon from the carbonyl, nitrile or sulfone group. See, for example, WO 92/16221, the disclosure of which is incorporated in its entirety herein by reference).

The activated linkers for attachment of glycol polymers (e.g., PEG polymers) to the protein can be monofunctional, bifunctional, or multifunctional.

Useful reagents having a reactive sulfone group that can be used in the methods include, without limitation, chlorosulfone, vinylsulfone and divinylsulfone.

In a specific embodiment, the glycol polymer is activated with a Michael acceptor. WO 95/13312, the disclosure of which is incorporated in its entirety herein by reference, describes, among other things, water soluble sulfone-activated PEGs which are highly selective for coupling with thiol moieties instead of amino moieties on molecules and on surfaces. These PEG derivatives are stable, against hydrolysis for extended periods in aqueous environments at pHs of about 11 or less, and can form linkages with molecules to form conjugates which are also hydrolytically stable. The linkage by which the PEGs and the biologically active molecule are coupled includes a sulfone moiety coupled to a thiol moiety and has the structure PEG-SO₂—CH₂—CH₂—S—W, where W represents the biologically active molecule, and wherein the sulfone moiety can be vinyl sulfone or an active ethyl sulfone. Two useful homobifunctional derivatives are PEG-bis-chlorosulfone and PEG-bis-vinylsulfone.

In one particularly useful embodiment, the protein is glycolated (e.g., PEGylated) by the coupling of a glycol polymer to the protein through glycosylations present on the protein (e.g., through an oligosaccharide structure of glycosylated asialic therapeutic protein i.e., having an N-linked oligosaccharide and produced in avian oviduct tissue such as chicken magnum tissue). Therefore, the invention includes proteins having glycol polymers such as polyethylene glycol coupled to a glycosylation structure of the glycosylated therapeutic protein and methods of making such glycosylated-glycolated protein.

In one specific embodiment, the invention is drawn to a process for the glycolation of a glycosylated protein, comprising activating a polyalkylene glycol, reacting the activated polyalkylene glycol with a diamino compound, whereby the activated polyalkylene glycol is coupled to the diamino compound through one of its amino groups, oxidizing a therapeutic protein to activate at least one glycosyl group therein, and reacting the polyalkylene glycol coupled to the diamino compound with the oxidized glycosyl group in the protein. For example, the invention can include a process for the PEGylation of a glycosylated protein comprising:
(a) reacting a polyethylene glycol of the formula CH₃O—(CH₂CH₂O)ₙ—H with o-nitrophenylchloroformate and triethylamine to produce a nitro compound of the formula CH₃O—(CH₂CH₂O)ₙ—COO-Ph-NO₂,
(b) reacting the nitro compound with a diaminoalkane of the formula H₂N—(CH₂)ₓ—NH₂ to produce an amino compound of the formula CH₃O—(CH₂CH₂O)ₙ—CO—NH—(CH₂)ₙ—NH₂,
(c) oxidizing sugar groups on the protein (e.g., glycosylated asialic EPO) to produce a macromolecule with an oxidized sugar residue, and
(d) reacting the amino compound with the activated macromolecule to produce a PEGylated molecule.

The result of this process is a PEGylated therapeutic protein, wherein PEG is bonded to the protein through an oligosaccharide structure, specifically, of the formula PEG-OCO—NH-alkylene-N═CH-glycosylated protein. Other aspects of this method of glycolating glycosylated proteins of the invention are disclosed in WO 94/05332, published Mar. 17, 1994, the disclosure of which is incorporated in its entirety herein by reference.

The invention can further be used to produce a wide range of desired glycolated and poultry derived therapeutic proteins (e.g., avian derived glycosylated therapeutic proteins) such as fusion proteins, growth hormones, cytokines, structural proteins and enzymes including human growth hormone, interferon, lysozyme, and β-casein. Other possible proteins contemplated for modification as disclosed herein include, but are not limited to, albumin, α-1 antitrypsin, antithrombin III, collagen, factors VIII, IX, X (and the like), fibrinogen, hyaluronic acid, insulin, lactoferrin, protein C, erythropoietin (EPO), granulocyte colony-stimulating factor (G-CSF), granulocyte macrophage colony-stimulating factor (GM-CSF), tissue-type plasminogen activator (tPA), somatotropin, and chymotrypsin. Modified immunoglobulins and antibodies, including immunotoxins which bind to surface antigens on human tumor cells and destroy them, can also be produced as disclosed herein.

Other examples of proteins which can be produced by transgenic avians and are contemplated for glycolation as disclosed herein include, without limitation, factor VIII, b-domain deleted factor VIII, factor VIIa, factor IX, anticoagulants; hirudin, alteplase, tpa, reteplase, tpa, tpa—3 of 5 domains deleted, insulin, insulin lispro, insulin aspart, insulin glargine, long-acting insulin analogs, hgh, glucagons, tsh, follitropin-beta, fsh, gm-csf, pdgh, ifn alpha2, ifn alpha2a, ifn alpha2b, inf-apha1, ifn-beta, inf-beta 1b, ifn-beta 1a, ifn-gamma (e.g., 1 and 2), il-2, il-11, hbsag, ospa, murine mab directed against t-lymphocyte antigen, murine mab directed against tag-72, tumor-associated glycoprotein, fab fragments derived from chimeric mab directed against platelet surface receptor gpII(b)/III(a), murine mab fragment directed against tumor-associated antigen ca125, murine mab fragment directed against human carcinoembryonic antigen, cea, murine mab fragment directed against human cardiac myosin, murine mab fragment directed against tumor surface antigen psma, murine mab fragments (fab/fab2 mix) directed against hmw-maa, murine mab fragment (fab) directed against carcinoma-associated antigen, mab fragments (fab) directed against nca 90, a surface granulocyte nonspecific cross reacting antigen, chimeric mab directed against cd20 antigen found on surface of b lymphocytes, humanized mab directed against the alpha chain of the il2 receptor, chimeric mab directed against the alpha chain of the il2 receptor, chimeric mab directed against tnf-alpha, humanized mab directed against an epitope on the surface of respiratory synctial virus, humanized mab directed against her 2, human epidermal growth factor receptor 2, human mab directed against cytokeratin tumor-associated antigen anti-ctla4, chimeric mab directed against cd 20 surface antigen of b lymphocytes domase-alpha dnase, beta glucocerebrosidase, tnf-alpha, il-2-diptheria toxin fusion protein, tnfr-lgg fragment fusion protein laronidase, dnaases, alefacept, darbepoetin alpha (colony stimulating factor), tositumomab, murine mab, alemtuzumab, rasburicase, agalsidase beta, teriparatide, parathyroid hormone derivatives, adalimumab (lgg1), anakinra, biological modifier, nesiritide, human b-type natriuretic peptide (hbnp), colony stimulating factors, pegvisomant, human growth hormone receptor antagonist, recombinant activated protein c, omalizumab, immunoglobulin e (ige) blocker, lbritumomab tiuxetan, ACTH, glucagon, somatostatin, somatotropin, thymosin, parathyroid hormone, pigmentary hormones, somatomedin, luteinizing hormone, chorionic gonadotropin, hypothalmic releasing factors, etanercept, antidiuretic hormones, prolactin and thyroid stimulating hormone.

The invention contemplates the modification of poultry derived immunoglobulins and other multimeric proteins in accordance with the invention. Examples of therapeutic antibodies that may be modified in methods of the invention include but are not limited to HERCEPTIN™ (Trastuzumab) (Genentech, CA) which is a humanized anti-HER2 monoclonal antibody for the treatment of patients with metastatic breast cancer; REOPRO™ (abciximab) (Centocor) which is an anti-glycoprotein IIb/IIa receptor on the platelets for the prevention of clot formation; ZENAPAX™ (daclizumab) (Roche Pharmaceuticals, Switzerland) which is an immunosuppressive, humanized anti-CD25 monoclonal antibody for the prevention of acute renal allograft rejection; PANOREX™ which is a murine anti-17-IA cell surface antigen IgG2a antibody (Glaxo Wellcome/Centocor); BEC2 which is a murine anti-idiotype (GD3 epitope) IgG antibody (ImClone System); IMC-C225 which is a chimeric anti-EGFR IgG antibody (ImClone System); VITAXIN™ which is a humanized anti-αVβ3 integrin antibody (Applied Molecular Evolution/MedImmune); Campath; Campath 1H/LDP-03 which is a humanized anti CD52 IgG1 antibody (Leukosite); Smart M195 which is a humanized anti-CD33 IgG antibody (Protein Design Lab/Kanebo); RITUXAN™ which is a chimeric anti-CD20 IgG1 antibody (IDEC Pharm/Genentech, Roche/Zettyaku); LYMPHOCIDE™ which is a humanized anti-CD22 IgG antibody (Immunomedics); ICM3 is a humanized anti-ICAM3 antibody (ICOS Pharm); IDEC-114 is a primate anti-CD80 antibody (IDEC Pharm/Mitsubishi); ZEVALIN™ is a radiolabelled murine anti-CD20 antibody (IDEC/Schering AG); IDEC-131 is a humanized anti-CD40L antibody (IDEC/Eisai); IDEC-151 is a primatized anti-CD4 antibody (IDEC); IDEC-152 is a primatized anti-CD23 antibody (IDEC/Seikagaku); SMART anti-CD3 is a humanized anti-CD3 IgG (Protein Design Lab); 5G1.1 is a humanized anti-complement factor 5 (CS) antibody (Alexion Pharm); D2E7 is a humanized anti-TNF-α antibody (CATIBASF); CDP870 is a humanized anti-TNF-α Fab fragment (Celltech); IDEC-151 is a primatized anti-CD4 IgG1 antibody (IDEC Pharm/SmithKline Beecham); MDX-CD4 is a human anti-CD4 IgG antibody (Medarex/Eisai/Genmab); CDP571 is a humanized anti-TNF-α IgG4 antibody (Celltech); LDP-02 is a humanized anti-α4β7 antibody (LeukoSite/Genentech); OrthoClone OKT4A is a humanized anti-CD4 IgG antibody (Ortho Biotech); ANTOVA™ is a humanized anti-CD40L IgG antibody (Biogen); ANTEGREN™ is a humanized anti-VLA-4 IgG antibody (Elan); CAT-152, a human anti-TGF-β₂ antibody (Cambridge Ab Tech); Cetuximab (BMS) is a monoclonal anti-EGF receptor (EGFr) antibody; Bevacizuma (Genentech) is an anti-VEGF human monoclonal antibody; Infliximab (Centocore, JJ) is a chimeric (mouse and human) monoclonal antibody used to treat autoimmune disorders; Gemtuzumab ozogamicin (Wyeth) is a monoclonal antibody used for chemotherapy; and Ranibizumab (Genentech) is a chimeric (mouse and human) monoclonal antibody used to treat macular degeneration.

In one embodiment, a poultry derived protein contemplated for modification as disclosed herein is an antibody capable of selectively binding to an antigen which may be generated by combining at least one immunoglobulin heavy chain variable region and at least one immunoglobulin light chain variable region, for example, cross-linked by at least one disulfide bridge. The combination of the two variable regions generates a binding site that binds an antigen using methods for antibody reconstitution that are well known in the art.

It is specifically contemplated that therapeutic proteins disclosed herein that do not normally or naturally contain an N-linked oligosaccharide can be produced recombinantly in the avian oviduct with an altered amino acid sequence such that the protein includes a site for an N-linked oligosaccharide, as is understood in the art. In addition, the proteins disclosed herein can be produced recombinantly in the avian oviduct such that additional sites for N-linked oligosaccharides are present on the protein, as is understood in the art.

In certain embodiments of the invention, the EPO (e.g., human EPO) not having the oligosaccharide structures required for useful biological activity is produced by heterologous gene expression in yeast (e.g., Pichia pastoris, Saccharomyces cerevisiae) for use in accordance with the invention. It is also contemplated that human EPO can be produced in bacteria and chemically glycosylated (e.g., E. coli, Bacillus sp.) for use in accordance with the present invention.

While it is possible that, for use in therapy, proteins of the invention may be administered in raw form, it can be useful to administer the protein as part of a pharmaceutical composition or pharmaceutical formulation.

The invention thus further provides pharmaceutical compositions comprising a glycolated protein of the invention or a pharmaceutically acceptable derivative thereof together with one or more pharmaceutically acceptable carriers thereof and, optionally, other therapeutic and/or prophylactic ingredients. The carrier(s) must be “acceptable” in the sense of being compatible with the other ingredients of the formulation and not deleterious to the recipient thereof.

Pharmaceutical compositions include those suitable for oral, rectal, nasal, topical (including buccal and sub-lingual), vaginal or parenteral (including intramuscular, sub-cutaneous and intravenous) administration or in a form suitable for administration by inhalation or insufflation. The formulations may, where appropriate, be conveniently presented in discrete dosage units and may be prepared by any of the methods well known in the art of pharmacy and medicine. All methods include the step of bringing into association the active compound with liquid carriers or finely divided solid carriers or both and then, if necessary, shaping the product into the desired formulation.

Pharmaceutical compositions suitable for oral administration may conveniently be presented as discrete units such as capsules, cachets or tablets each containing a predetermined amount of the active ingredient; as a powder or granules; as a solution; as a suspension; or as an emulsion. The active ingredient may also be presented as a bolus, electuary or paste. Tablets and capsules for oral administration may contain conventional excipients such as binding agents, fillers, lubricants, disintegrants, or wetting agents. The tablets may be coated according to methods well known in the art. Oral liquid preparations may be in the form of, for example, aqueous or oily suspensions, solutions, emulsions, syrups or elixirs, or may be presented as a dry product for constitution with water or other suitable vehicle before use. Such liquid preparations may contain conventional additives such as suspending agents, emulsifying agents, non-aqueous vehicles (which may include edible oils) or preservatives.

The compounds according to the invention may also be formulated for parenteral administration (e.g., by injection, for example bolus injection or continuous infusion) and may be presented in unit dose form in ampoules, pre-filled syringes, small volume infusion or in multi-dose containers with an added preservative. The compositions may take such forms as suspensions, solutions, or emulsions in oily or aqueous vehicles, and may contain formulatory agents such as suspending, stabilizing and/or dispersing agents. Alternatively, the active ingredient may be in powder form, obtained by aseptic isolation of sterile solid or by lyophilization from solution, for constitution with a suitable vehicle, e.g., sterile, pyrogen-free water, before use.

For topical administration to the epidermis, the compounds according to the invention may be formulated as ointments, creams or lotions, or as a transdermal patch. Ointments and creams may, for example, be formulated with an aqueous or oily base with the addition of suitable thickening and/or gelling agents. Lotions may be formulated with an aqueous or oily base and will in general also contain one or more emulsifying agents, stabilizing agents, dispersing agents, suspending agents, thickening agents or coloring agents.

Formulations suitable for topical administration in the mouth include lozenges comprising active ingredient in a flavored base, usually sucrose and acacia or tragacanth; pastilles comprising the active ingredient in an inert base such as gelatin and glycerin or sucrose and acacia; and mouthwashes comprising the active ingredient in a suitable liquid carrier.

Pharmaceutical compositions suitable for rectal administration wherein the carrier is a solid, are most preferably represented as unit dose suppositories. Suitable carriers include cocoa butter and other materials commonly used in the art, and the suppositories may be conveniently formed by a mixture of the active compound with the softened or melted carrier(s) followed by chilling and shaping in molds.

Formulations suitable for vaginal administration may be presented as pessaries, tampons, creams, gels, pastes, foams or sprays containing in addition to the active ingredient, such carriers as are known in the art to be appropriate.

For intra-nasal administration the compounds of the invention may be used as a liquid spray or dispersible powder or in the form of drops.

Drops may be formulated with an aqueous or non-aqueous base also comprising one or more dispersing agents, solubilizing agents or suspending agents. Liquid sprays are conveniently delivered from pressurized packs.

For administration by inhalation, the compounds according to the invention are conveniently delivered from an insufflator, nebulizer or a pressurized pack or other convenient means of delivering an aerosol spray. Pressurized packs may comprise a suitable propellant such as dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount.

Alternatively, for administration by inhalation or insufflation, the compounds according to the invention may take the form of a dry powder composition, for example a powder mix of the compound and a suitable powder base such as lactose or starch. The powder composition may be presented in unit dosage form in, for example, capsules or cartridges or, e.g., gelatin or blister packs from which the powder may be administered with the aid of an inhalator or insufflator.

When desired, the above described formulations adapted to give sustained release of the active ingredient, may be employed.

The pharmaceutical compositions according to the invention may also contain other active ingredients such as antimicrobial agents, or preservatives.

In addition, it is contemplated that the compounds of the invention may be used in combination with other therapeutic agents. For example, poultry derive glycosylated-glycolated human interferon alpha (e.g., interferon alpha 2b) can be used in combination with ribavirin and/or virimidine to treat viral infections such as hepatitis C.

Compositions or compounds of the invention can be used to treat a variety of conditions. For example, there are many conditions for which treatment therapies are known to practitioners of skill in the art in which protein therapeutics such as erythropoietin are employed. The present invention contemplates that the protein therapeutics produced in accordance with the invention can be employed to treat such conditions. That is, the invention contemplates the treatment of a condition known to be treatable by a protein therapeutic such as EPO by administering a protein therapeutic produced in accordance with the invention, such as glycolated asialic EPO.

Generally, the dosage administered will vary depending upon known factors such as age, health and weight of the recipient, type of concurrent treatment, frequency of treatment, and the like. Usually, a dosage of active ingredient can be between about 0.0001 and about 10 milligrams per kilogram of body weight. Precise dosage, frequency of administration and time span of treatment can be determined by a physician skilled in the art of therapeutic protein administration.

The following examples are methods useful for the production of therapeutics in accordance with the present invention; however, it is understood that the invention is not limited to any particular method of making therapeutics of the invention and the invention encompasses all such useful methods known in the art and those yet to be devised.

### EXAMPLE 1

### Bioactivity Determination of Trangeneic Poultry Derived Human EPO by Cell Proliferation Assay

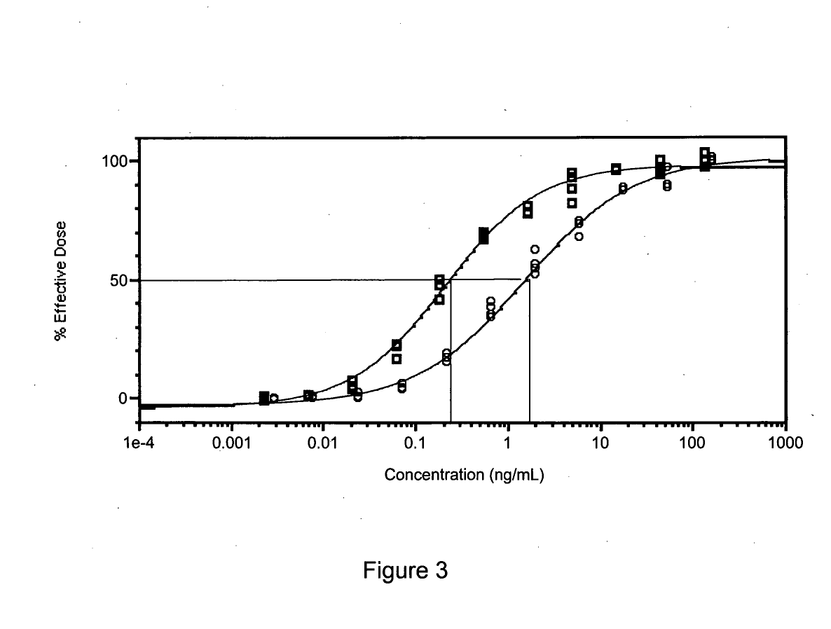

The biological activity of poultry derived EPO was assessed by an in vitro cell proliferation assay that quantifies the effect of EPO on Human Erythroleukemia (TF-1) cells. A comparison of the activity for transgenic poultry derived human EPO (TPD EPO) and human EPO obtained from recombinant CHO cells (CHO EPO) was determined and is shown in FIG. 3.

The cells were grown in RPMI-1640 medium with 2 mM L-glutamine, 1 mM sodium pyruvate, 50 mM 2-mercaptoethanol, 2 ng/ml rhGM-CSF and 10% fetal bovine serum. For 24-48 hr preceding the assay the cells were cultured in 5 ng/ml GM-CSF. The cells were then washed 3 times with cold RPMI and re-suspended in cell culture medium (without GM-CSF). The cell density of the suspension was adjusted to 1×10⁵ cells/ml and 100 ul of the cell suspension was added to each well in a 96-well assay plate.

In a second 96 well plate, 100 uL of TPD EPO (160 ng/ml) was added to the first column of wells and then diluted, three fold per dilution, in each succeeding well in each row to produce a serial dilution of the EPO. 20 ul of TPD EPO from each serially diluted well was added to the corresponding well in the assay plate containing the cells.

The assay plate was incubated at 37° C. in 5% CO₂ for 5 days at which time an MTT-assay was performed which is based on the principle that metabolically active cells are able to reduce 3-(4,5-dimethylthiazol-2,5-diphenyl)tetrazolium bromide (MTT) to a colored product, which can be quantified spectrophotometrically. The same assay was performed using CHO EPO. The optical density (OD) was determined for each EPO concentration for both the TPD EPO plate and CHO EPO plate. The assays were performed in triplicate for each EPO and the ED₅₀ was determined as shown in FIG. 3. The ED₅₀ of TPD EPO was shown to be approximately 7 times that of CHO EPO.

### EXAMPLE 2

### Preparation of TPD EPO Conjugated to Linear MPEG-SC-20 KDa and PK Determination

A 5.0 mM stock solution of MPEG-SC-20 KDa, purchased from Laysan Bio, Inc, Arab, Ala., was prepared in acetonitrile. A 4.7 μM stock solution of purified TPD EPO was prepared in conjugation buffer. The conjugation reaction was initiated by mixing 5 ml of the TPD EPO stock with 2.4 ml of conjugation buffer followed by the addition of 400 μl of the MPEG-SC-20 KDa stock solution resulting in a PEG:EPO molar ratio of about 85:1. The reaction was allowed to proceed overnight at room temperature. To stop the reaction, glycine was added to the reaction mix to a concentration of 20 mM, and the mix was allowed to stand for 20 minutes at room temperature. The final volume of the PEG-EPO conjugation mix was about 7.8 ml, containing about 96 μg/ml EPO.

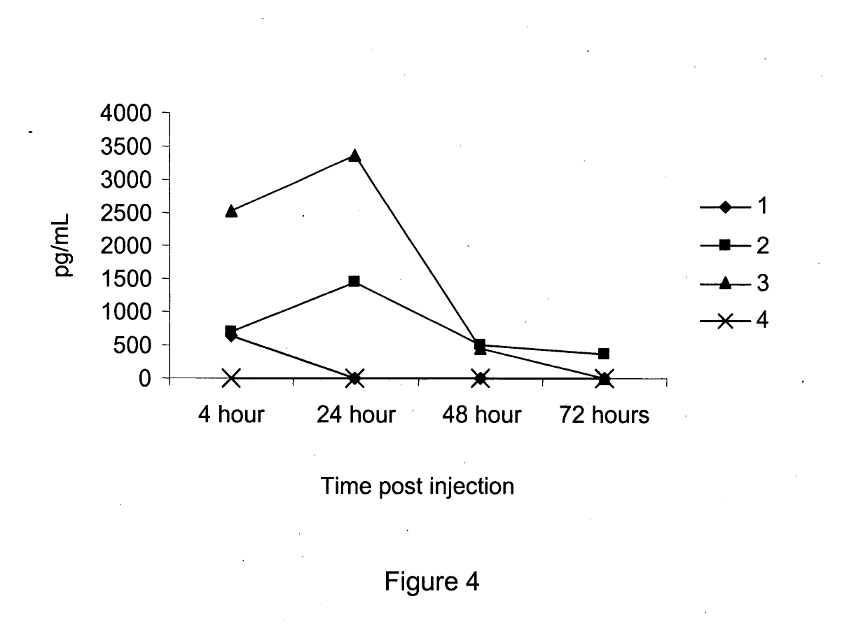

For PK measurements approximately 200 IU of each EPO (16.7 μg of EPO) was injected into a rat. Blood samples were taken from the rat using standard methodologies at 4 h, 24 h, 48 h and 72 h time points. Serum was harvested from the blood samples, diluted 1:10 and EPO was quantified in pg/ml by ELISA using a human erythropoietin immunoassay kit (StemCell Technologies, Inc). For each EPO type the assays were performed in 3 rats (assays were performed in duplicate at each time point for each rat and averaged) and averages for the three rats are shown graphed. The PK measurement for PEG conjugated TPD EPO is shown as line “2” in FIG. 4 (20 KDa PEG). Line “4” in FIG. 4 shows the measured PK for non-PEGylated TPD EPO and line “3” shows the measured PK for EPO produced in CHO cells.

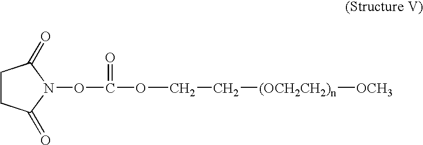

Above is shown MPEG-SC-20 KDa (average molecular weight of 20 KDa). This PEG structure also represents the activated PEG molecule employed in Example 3, having an average molecular weight of 5 KDa. The PEG molecule is disclosed in one or more of U.S. Pat. No. 5,122,614, issued Jun. 16, 1992; U.S. Pat. No. 5,612,460, issued Mar. 18, 1997; U.S. Pat. No. 6,602,498, issued Aug. 5, 2003; U.S. Pat. No. 6,774,180, issued Aug. 10, 2004; and US patent publication No. 2006/0286657, published Dec. 21, 2006. The disclosures of each of these four issued patents and one published patent application are incorporated in their entirety herein by reference.

### EXAMPLE 3

### Preparation of TPD EPO Conjugated with Linear MPEG-SC-5 KDa and PK Determination

A 2.0 mM stock solution of MPEG-SC-5 KDa, purchased from Laysan Bio, Inc, Arab, Ala., was prepared in acetonitrile. A 4.7 μM stock solution of TPD EPO was prepared in conjugation buffer. The reaction was initiated by mixing 5 ml of EPO stock with 588 μl of conjugation buffer and then adding 294 μl of the PEG stock solution resulting in PEG:EPO molar ratio of about 25:1. The reaction was allowed to proceed over night at room temperature. To stop the reaction, glycine was added to the reaction mix to a concentration of 20 mM, and the mix was allowed to stand for 20 minutes at room temperature. The final volume of the PEG-EPO conjugation mix was about 5.88 ml, containing about 129 μg/ml EPO.

For PK measurements approximately 200 IU of each EPO (16.7 μg of EPO) was injected into a rat. Blood samples were taken from the rat using standard methodologies at 4 h, 24 h, 48 h and 72 h time points. Serum was harvested from the blood samples, diluted 1:10 and EPO was quantified in pg/ml by ELISA using a human erythropoietin immunoassay kit (StemCell Technologies, Inc). For each EPO type injected, the assays were performed in 3 rats (assays were performed in duplicate at each time point for each rat and averaged) and averages for the three rats are shown graphed. The PK measurement for PEG conjugated TPD EPO is shown as line “1” in FIG. 4 (5 KDa PEG). Line “4” in FIG. 4 shows the measured PK for non-PEGylated TPD EPO and line “3” shows the measured PK for EPO produced in CHO cells.

### EXAMPLE 4

### Preparation of EPO Conjugated with 40 KDa NHS Y-PEG and PK Determination

A 6.7 mM stock solution of 40 KDa NHS Y-PEG, purchased from JemKem Technology USA, was prepared in acetonitrile. A 10.4 μM stock solution of TPD EPO was prepared in 100 mM phosphate buffer pH adjusted to 7.0. The conjugation reaction was initiated by mixing 4 ml of EPO stock with 4 ml of 100 mM phosphate buffer, pH 7.0 and then adding 500 μl of the PEG stock solution resulting in PEG:EPO molar ratio of about 80:1. The reaction was allowed to proceed 2 to 4 h at room temperature. To stop the reaction, glycine was added to the reaction mix to a concentration of 20 mM, and the mix was allowed to stand for 20 minutes at room temperature. The final reaction volume was 8.5 ml.

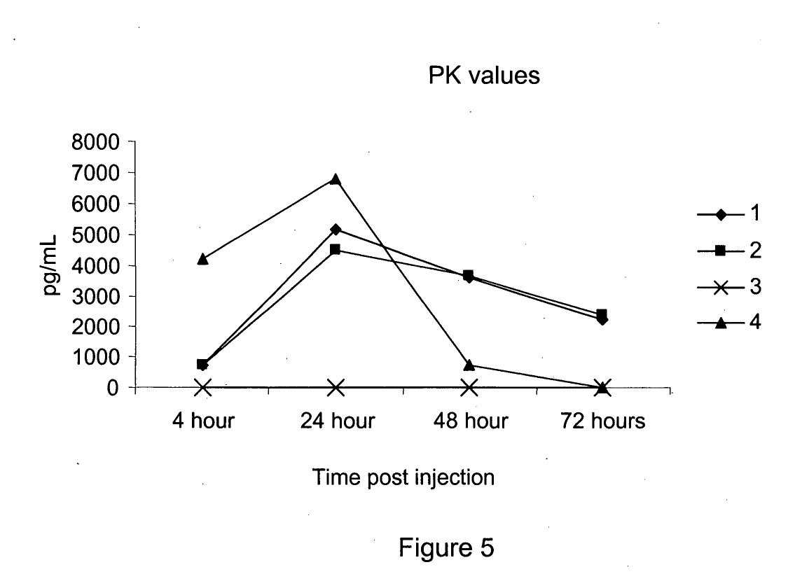

For PK measurements approximately 200 IU of each EPO (16.7 μg of EPO) was injected into a rat. Blood samples were taken from the rat using standard methodologies at 4 h, 24 h, 48 h and 72 h time points. Serum was harvested from the blood samples, diluted 1:10 and EPO was quantified in pg/ml by ELISA using a human erythropoietin immunoassay kit (StemCell Technologies, Inc). The assays for each EPO type were performed in 3 rats (assays were performed in duplicate at each time point for each rat and averaged) and averages for the three rats are shown graphed. The PK measurement for PEG conjugated TPD EPO is shown as line “2” in FIG. 5 (40 KDa PEG). Line “3” in FIG. 5 shows the measured PK for non-PEGylated TPD EPO and line “4” shows the measured PK for EPO produced in CHO cells.

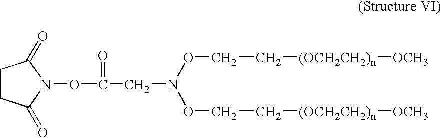

Above is shown the 40 KDa NHS Y-PEG (average molecular weight of 40 KDa). This PEG molecule is disclosed in US patent publication No. 2005/0180946, published Aug. 18, 2005, the disclosure of which is incorporated in its entirety herein by reference.

### EXAMPLE 5

### Preparation of EPO Conjugated with Linear GLUC-NHS-35 KDa PEG and PK Determination

A 5.7 mM stock solution of GLUC-NHS-35 KDa PEG was prepared in acetonitrile. A 10.4 μM stock solution of TPD EPO was prepared in 100 mM phosphate buffer pH adjusted to 7.0. The conjugation reaction was initiated by mixing 2.5 ml of EPO stock with 2.5 ml of 100 mM Phosphate buffer, pH 7.0 and then adding 400 μl of the PEG stock solution resulting in approximate molar ratio of 85 PEG/EPO. The reaction was allowed to proceed about 2 h at room temperature. To stop the reaction, glycine was added to the reaction mix to a concentration of 20 mM, and the mix was allowed to stand for 20 minutes at room temperature. The final reaction volume was 5.4 ml.

For PK measurements approximately 200 IU of each EPO (16.7 μg of EPO) was injected into a rat. Blood samples were taken from the rat using standard methodologies at 4 h, 24 h, 48 h and 72 h time points. Serum was harvested from the blood samples, diluted 1:10 and EPO was quantified in pg/ml by ELISA using a human erythropoietin immunoassay kit (StemCell Technologies, Inc). The assays for each EPO type were performed in 3 rats (assays were performed in duplicated at each time point for each rat and averaged) and averages for the three rats are shown graphed. The PK measurement for conjugated TPD EPO is shown as line “1” in FIG. 5 (GLUC-NHS-35 KDa PEG). Line “3” in FIG. 5 shows the measured PK for non-PEGylated TPD EPO and line “4” shows the measured PK for fully sialated EPO produced in CHO cells.

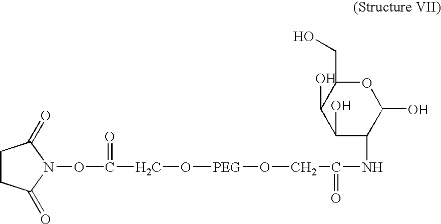

Above is shown the GLUC-NHS-35 KDa PEG (average molecular weight of 35 KDa). This PEG molecule is disclosed in US patent publication No. 2006/0014666, published Jan. 19, 2006, the disclosure of which is incorporated in its entirety herein by reference. This PEG is particularly useful in accordance with the invention and is specifically contemplated for use with each transgenic avian (e.g., poultry such as chicken) derived therapeutic protein disclosed herein.

### EXAMPLE 6

### Hematocrit Analysis of TPD EPO Conjugated with 40 KDa NHS V-PEG and TPD EPO Conjugated to Linear GLUC-NHS-35 KDa PEG

EPO conjugated with 40 KDa NHS Y-PEG of Example 4 and the EPO conjugated with the linear GLUC-NHS-35 KDa PEG of Example 5 were each separated from non-pegylated EPO (to greater than 90% purity) by chromatography using a 5 ml HiTrap® SPFF column (GE Life Sciences). Briefly, an elution gradient was run in 50 mM NaOAc (pH 4.0) with a starting NaCl concentration of 0 M run to a final concentration of 0.5 M. The PEGylated EPO eluted at about 0.35 M NaCl and the non-PEGylated at about 0.5 M.

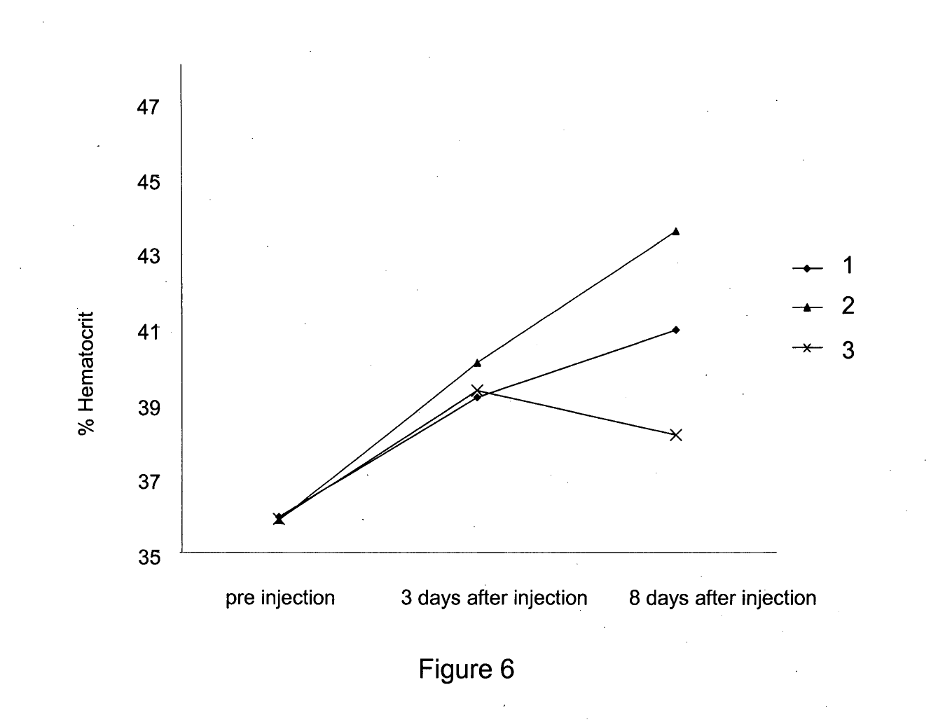

PD (hematocrit) was measured after injecting 16.7 μg (200 IU) of each purified conjugated EPO and the unconjugated EPO subcutaneously into rats of approximately 250 grams. The data shows the hematocrit levels of blood samples collected at day 0, day 3 and day 8 post injection. Relative hematocrit levels were measured using standard procedures where the blood samples were centrifuged in a capillary tube and the packed red blood cell volume was quantified. Each data point on the graph in FIG. 6 represents the average of 3 independent hematocrit assays for each of four injected rats.

The graphed data in FIG. 6 shows the increase in hematocrit for TPD EPO conjugated to linear GLUC-NHS-35 KDa PEG and for TPD EPO conjugated to 40 KDa NHS Y-PEG (line 1 and line 2 respectively). Both PEGylated forms of the EPO show a substantially higher hematocrit than for the unpegylated TPD EPO (line 3). The apparent increase in hematocrit for the non-pegylated TPD EPO is likely due to the expected hematocrit increase for rats of less than one year of age which were used in the study.

### EXAMPLE 7

### Preparation of O-PEG-(P-Azo Poultry Derived Glycosylated Factor VIII benzyl)ether

Formation of O-PEG-p-amino benzyl ether

3.46 g of p-Nitrobenzyl chloride, 2.0 g of powdered sodium hydroxide, 20 ml of anhydrous tetrahydrofuran and 0.01 mole of PEG are refluxed for 3 hours. The solution is filtered and evaporated under reduced pressure and PEG-p-nitronenzyl ether is precipitated by the addition of petroleum ether (bp 30° C. to 40° C.). The nitro ether is reduced with hydrogen at atmospheric pressure in the presence of Raney nickel catalyst (about 1 g) in ethanol (50 ml). The catalyst is removed and the filtrate evaporated yielding O-PEG-p-amino benzyl ether.

Coupling with Factor VIII

O-PEG-p-amino benzyl ether is diazotized in aqueous solution at 0° C. with nitrous acid. To the purified diazotized solution an aqueous solution of 0.25% glycosylated Factor VIII, produced as disclosed in U.S. Pat. No. 6,730,822, issued May 4, 2004, is added and the mixture is kept at 0° C. for 2 hours. The solution is dialysed at 5° C. to 10° C. to yield glycosylated-PEGylated Factor VIII.

### EXAMPLE 8

### Preparation of O-Peg Methyl Carboxy Poultry Derived Human GM-CSF

### Preparation of PEG-Methyl Carbomethoxy Ester

2.0 g of PEG 750 is dissolved in 30 ml of liquid ammonia and the solution is treated with sodium until a blue color persists for 5 minutes. The ammonia is allowed to evaporate on a stream of dry nitrogen. The residue is treated with 5 ml of methyl chloroacetate and the mixture is allowed to stand overnight at room temperature followed by heating to 100° C. for 1 hour. The excess reagent is removed under reduced pressure to provide PEG-methyl carbomethoxy ester.

### Activation of PEG

To a solution of 1.0 g of O-PEG-methyl carbomethoxy ester in 10 ml water a solution of 1.0 g of N-ethoxycarbonyl-2-ethoxy 1,2-dihydroquinoline (EEDQ) in 10 ml of 10% acetone is added dropwise. The pH is maintained at 7.0 and after 30 minutes, the pH is adjusted to 1.0 with concentrated hydrochloric acid and is maintained at this pH for 90 seconds to destroy excess EEDQ. The pH of the solution is then adjusted to pH 8.

### Coupling to GM-CSF

50 mg of human glycosylated GM-CSF produced as disclosed in U.S. Pat. No. 6,730,822, issued May 4, 2004, in phosphate buffer, pH 8.0, is added to the solution of the activated PEG at 4° C. to 5° C. After ½ hour the solution is dialyzed against water yielding N-glycosylated-pegylated poultry derived GM-CSF.

### EXAMPLE 9

### Preparation of 1-(Poultry Derived Human Interferon beta1b-2-hydroxy propoxy)-4-3″-O-PEG-2″-hydroxy propoxy Butane

### Oxirane Ether of PEG

5.0 g of PEG, 1 ml of 1,4-butanediol diglycidyl ether and 1 ml of 0.6 M sodium hydroxide solution containing 2 mg of sodium borohydride are stirred at room temperature for 8 hours. The solution is neutralized and evaporated. The residue is extracted with acetone and the PEG ether precipitated by the addition of excess petroleum ether.

Coupling to Interferon beta1b

1.0 g of oxirane-PEG and 50 mg of human interferon beta1b produced as disclosed in U.S. Pat. No. 6,730,822, issued May 4, 2004, in buffer solution (pH 8.5) are allowed to react at room temperature for 48 hours. The solution is dialyzed to yield poultry derived N-glycosylated-PEGylated interferon beta1b.

### EXAMPLE 10

### Preparation of PEGylated, Glycosylated Poultry Derived Human FSH

Activation of Methoxy-PEG (mPEG)

Two grams of 15 KDa mPEG (0.1 mM, final concentration), is dissolved in 20 ml of acetonitrile with 0.24 g of o-nitrophenylchloroformate (1.2 mM) and 33 microliters of triethylamine (1.2 mM) and is stirred for 24 hours at room temperature.

The triethylammonium chloride is then filtered off using a sintered glass funnel. 200 ml of ethyl ether is added, and the solution is left to crystallize overnight at 4° C. The product is filtered, washed with ether to remove all of the yellow color, and recrystallized from acetonitrile-ether. The product is then assayed spectrophotometrically by the release of p-nitrophenol by ε-amino-n-caproic acid (ACA).

### PEGylation of Poultry Derived Human FSH Through Lysine Groups

5 mg of poultry derived human FSH, produced as disclosed for other pharmaceutical proteins in U.S. Pat. No. 6,730,822, issued May 4, 2004, is dialyzed extensively into 50 mM sodium borate buffer pH 8.3.

To the dialyzed FSH sample, activated mPEG is added to a 5 fold molar excess and the mix is incubated at room temperature with shaking for 30 minutes. The reaction is stopped by loading the sample on a NAP 25 (Pharmacia) desalting column and eluting it with 50 mM NaPO₄ buffer, pH 6.8. The desalted sample is loaded on Superose 6 column (1×30 cm BioRad Econocolumn®) and eluted with 50 mM NaPO₄ buffer, pH 6.8. Resultant peaks from the Superose column are assayed by SDS-PAGE and pooled.

### EXAMPLE 11

### Preparation of Poultry Derived Human Follicle Stimulating Hormone (FSH) Having PEG coupled to a Glycosylation Structure Present on the FSH

Making the Amino Derivative of mPEG-μ-pNP (PEG-μ-butamine)

0.5 g of mPEG-μ-p-nitrophenyl is slowly added to 5 ml of 50 mM Na-borate buffer, pH 9.0, containing 44.25 mg (100 mmoles) of 1,4-aminobutane. The reaction is incubated at room temperature with shaking for 3 hours. The reaction is stopped by passing it through an NAP 25 desalting column and eluted with water and dialyzed into milli-Q H₂O. The dialyzed material is lyophylized and weighed.

### Oxidation of Poultry Derived Human FSH

| | |
|---|---|
| Coupling Buffer: | 0.05 M sodium acetate |
| | 0.1 M sodium chloride, pH 5.0 |
| Wash Buffer: | 0.1 M sodium acetate |
| | 0.5 M sodium chloride, pH 3.5 |
| Storage Buffer: | 0.05 M sodium phosphate, pH 6.8 |

0.5 mg of poultry derived human FSH is buffer exchanged into the coupling buffer using an NAP-10 (Pharmacia) desalting column. To the poultry derived protein solution is added 0.1 ml of freshly prepared 100 mM sodium m-periodate (NaIO₄). The solution is mixed gently, and the sealed reaction vessel is shielded from light and incubated at room temperature for 30 minutes. To stop the reaction, the sample is passed through a NAP-10 desalting column and is equilibrated with wash buffer. The column is eluted with the conjugation buffer.

Coupling of Oxidized Poultry Derived Human FSH to PEG-μ-butamine

To the desalted, oxidized poultry derived human FSH is added 5 mg of PEG-μ-butamine. The reaction mix is overlayed with nitrogen and is tumbled gently overnight at 4° C. The molar ratio of poultry derived human FSH to PEG-μ-butamine is 1:100. The sample is then loaded onto a Superose 6 column. The FSH containing peaks are pooled and are concentrated on an amicon stirred cell concentrator.

All documents (e.g., U.S. patents, U.S. patent applications, publications) cited in the above specification are incorporated herein by reference. Various modifications and variations of the present invention will be apparent to those skilled in the art without departing from the scope and spirit of the invention. Although the invention has been described in connection with specific preferred embodiments, it should be understood that the invention as claimed should not be unduly limited to such specific embodiments. Various modifications of the described modes for carrying out the invention which are obvious to those skilled in the art are intended to be within the scope of the following claims.

What is claimed is:

1. A therapeutic protein comprising an N-linked oligosaccharide wherein the N-linked oligosaccharide substantially excludes sialic acid and the therapeutic protein is chemically bonded to a glycol polymer.

2. The therapeutic protein of claim 1 comprising an O-linked oligosaccharide.

3. The therapeutic protein of claim 1 wherein two N-linked oligosaccharides exclude a sialic acid.

4. The therapeutic protein of claim 1 wherein three N-linked oligosaccharides exclude a sialic acid.

5. The composition of claim 1 wherein the glycol polymer is a polyalkylene glycol.

6. The therapeutic protein of claim 1 wherein the glycol polymer is polyethylene glycol.

7. The therapeutic protein of claim 1 wherein the glycol polymer has a molecular weight between about 300 KDa and about 200,000 KDa.

8. The composition of claim 1 wherein the glycol polymer is covalently bonded to at least one of an amino group, a hydroxyl group, a sulfhydryl group and a carboxyl group of the therapeutic protein.

9. The therapeutic protein of claim 1 obtained from a transgenic avian.

10. The composition of claim 9 wherein the avian is selected from the group consisting of chicken, turkey and quail.

11. The composition of claim 9 wherein the avian is a chicken.

12. The composition of claim 9 wherein the therapeutic protein is produced in a tubular gland cell.

13. The composition of claim 1 wherein the therapeutic protein excludes a terminal sialic acid.

14. The therapeutic protein of claim 1 wherein the therapeutic protein is selected from the group consisting of EPO, Factor VIII, Factor VIIa, Factor IX, alteplase tPA, Reteplase tPA (differs from h tPA—3 of 5 domains deleted), growth hormone (e.g., hGH), thyroid stimulatin hormone (TSH), follicle stimulating hormone (FSH), follitropin-beta, calcitonin, platelet derived growth factor (PDGF), keratinocyte growth factor, insulin-like growth factor-1 (IGF-1), IGFBP-3, GM-CSF, INF-beta, INF-beta1b and IFN-gamma, IFN-gamma 1b, IL-3 and IL-12, TNFR-IgG fragment fusion protein, beta glucocerebrosidase, asparaginase, urokinase, adenosin deaminase, agalsidase alfa, idursulfase, alpha-L-iduronidase, galsulfase: arylsulfatase, galsulfase: arylsulfatase B, BM 102, N-acetylgalactosamine-4-sulfatase, hASB, activated protein C, dornase-alpha DNAse, anakinra, eptotermin alfa, Protein C and dibotermin alfa.

15. The therapeutic protein of claim 1 wherein the therapeutic protein is erythropoietin.

16. The composition of claim 15 wherein the erythropoietin is human erythropoietin.

17. The therapeutic protein of claim 1 wherein the therapeutic protein has the amino acid sequence set forth in FIG. 2B.

18. A composition comprising an asialic therapeutic protein obtained from a transgenic avian covalently bonded to a glycol polymer.

19. The composition of claim 18 wherein the therapeutic protein is erythropoietin.

20. The composition of claim 19 wherein the amino acid sequence of the erythropoietin is set forth in FIG. 2B.

21. The composition of claim 18 wherein the therapeutic protein is produced in an oviduct cell of the transgenic avian.

22. The composition of claim 21 wherein the oviduct cell is a tubular gland cell.

23. The composition of claim 18 wherein the glycol polymer is a polyethylene glycol.

24. The composition of claim 18 wherein the glycol polymer has a molecular weight of about 300 to about 200,000.

25. A method comprising,
producing an therapeutic protein which excludes sialic acid being terminally linked to an oligosaccharide of the therapeutic protein; and covalently bonding a glycol polymer to the therapeutic protein.

26. A pharmaceutical composition comprising erythropoietin according claim 25.

27. A method of treating a condition comprising administering to a patient in need thereof a pharmaceutical composition of claim 26.
